(19) United States
(12) Patent Application Publication
(10) Pub. No.: US 2007/0092479 A1
(43) Pub. Date: Apr. 26, 2007

(54) Odor mitigation compositions, devices, and methods

(75) Inventor: Scott J. Turriff, De Pere, WI (US)

Correspondence Address: WILHELM LAW SERVICE, S.C., 100 W LAWRENCE ST, THIRD FLOOR, APPLETON, WI, 54911, US

(73) Assignee: Natural Predator, LLC, De Pere, WI (US)

(21) Appl. No.: 11/585,072

(22) Filed: Oct. 23, 2006

Related U.S. Application Data
(60) Provisional application No. 60/762,250, filed on Jan. 25, 2006.
(60) Provisional application No. 60/729,014, filed on Oct. 21, 2005.
(60) Provisional application No. 60/773,849, filed on Feb. 15, 2006.

(51) Int. Cl. A61L 9/00

(52) U.S. Cl. 424/76.2

(57) ABSTRACT

Methods, devices, and corresponding compositions for generally simultaneously camouflaging and suppressing odors by applying at least one odor absorbing agent, optionally a plurality of differently colored odor absorbing agents, directly to a user and/or to garments and/or accessories used in hunting and/or observing and/or photographing wild animals. The odor absorbing agents are applied to such user and/or to garments and/or accessories in a manner which facilitates a reduction in the relative visual conspicuousness of the user.

### BACKGROUND

This invention relates generally to odor or aroma suppressing, absorbing, eliminating, and/or otherwise mitigating substances, as well as pigmented and/or otherwise colored odor or aroma suppressing, absorbing, eliminating, and/or otherwise mitigating substances. In addition, this invention relates to methods of applying and/or reapplying, odor or aroma suppressing, absorbing, eliminating, and/or otherwise odor mitigating substances and compounds for use in outdoor recreational activities, such as hunting, game observation which includes game or wilderness photography, and/or others.

Various outdoor recreational activities, such as animal, e.g. game, hunting and/or observing are, to some individuals, enjoyable and popular activities. Some hunting and observing activities tend to yield relatively more successful results when a hunter or observer is in relatively close proximity to the game which is hunted or observed. Exemplary of such activities are (i) large-game or big-game hunting, and (ii) large-game or big-game observation e.g. watching, video recording, and/or photographing.

Many game animals, and other animals, instinctively fear, or are apprehensive of, human beings. Correspondingly, when enjoying outdoor recreational activities, such as game animal hunting or game or other animal observation, the hunter or observer can find it relatively difficult to sufficiently approach the game, or remain in close enough proximity to the game for a sufficient period of time, before the game runs off or otherwise evades observation.

Many big-game animals which include, for example, various deer such as white-tailed deer, mule deer, caribou, elk, moose, and others, various bears such as black bears, brown bears, and others, various canines such as fox, wolves, coyotes, and others, have relatively sensitive olfactory systems. Accordingly, many of such game animals can effectively detect the presence of, for example, a hunter or observer by detecting the odor or aroma of the hunter or observer, whereby approaching such big-game animals or staying within a relatively close proximity of such big-game animals can prove relatively difficult.

Attempts have been made to provide products, for use during outdoor recreational activities, which are applied to the bodies of users, which are supposed to mask human scents. Other attempts to address human odor problems during big-game hunting and observing include wearing articles of clothing which include odor absorbing layers. And yet other attempts to address human odor problems during big-game hunting and observing include spray, e.g. pump-spray, applying various liquid products to clothing.

In addition, many game animals have relatively sensitive sight organs and systems. Accordingly, many of such big-game animals can effectively detect the presence of, for example, a hunter or observer by detecting visual stimulus, whereby such big-game animals are capable of seeing hunters or observers. Correspondingly, big-game hunters or big-game observers which are relatively more visually conspicuous, tend to find it relatively more difficult to approach, or stay within a relatively close proximity of, big-game animals for sufficient periods of time.

In some game hunting and observing situations, the hunter or observer attempts to make his/her body relatively less visually conspicuous by way of, for example, camouflage or other visual masking techniques and/or products. However, users have a limited number of choices in selecting odor absorbing products, many of which can compromise the integrity of the desired camouflage or other visual masking techniques and/or products, by way of e.g. smutting, or otherwise reducing the effectiveness of the desired camouflage or other visual masking techniques and/or products.

However, articles of clothing which are designed to be odor absorbing are sometimes not desirable. Namely, the odor absorbing layers of the odor absorbing articles of clothing lose odor absorbing effectiveness over time. Such articles of clothing can sometimes be recharged by, for example, running the clothing through a clothes dryer cycle. However, such garments can not be recharged in such a manner indefinitely. Thus such odor absorbing articles of clothing enjoy only a limited odor absorbing use life.

Spray delivery systems for applying odor absorbing liquids are sometimes not desirable. As one example, when a user applies an odor absorbing liquid to articles of clothing, the user must preferably spray the formulation so as to cover substantially the entire surface area of the clothing articles. Failure to do so might enable human odors to emanate through the untreated portion of the article of clothing.

As another example, delivery orifices of the hand-pump-spray bottles can clog or become obstructed by various odor absorbing particles suspended in the liquid odor absorbing formulation. This is especially likely with hand-pump-spray bottles since typically a relatively low pressure is generated by the manually actuated device, whereby accumulation of particles at or adjacent the delivery orifices can occur.

Accordingly, when odor absorption treating clothing, since the user must apply the odor absorbing liquid to a sufficiently large portion of the surface area of the clothing, and since the user might occasionally have to clear clogs or obstructions from the orifices of the pump-spray bottle, spray application of odor absorbing liquid formulas can be relatively time consuming.

Some odor absorbing products produce non-desired smutting effects or other visual defects upon the surface of the clothing to which it is applied. Such products can also soil the skin of a user upon direct contact.

Accordingly, it is desirable to provide garments or other game hunting or game observing accessories with removable portions thereof, wherein the removable portions are adapted and configured to accept, as first applications or reapplications, odor absorbing products thereupon.

It is also desirable to provide garments with odor absorbing removable layers, which are housed between an inner layer and an outer layer, whereby the visual integrity of the outer surface of the outer layer is maintained and the user's skin is generally not in direct contact with the odor absorbing removable layer.

It is beneficial to provide odor absorbing products which mitigate the visually detrimental effects typically associated with applying odor absorbing products to e.g. camouflage garments and/or the skin of the user.

It is also beneficial to provide odor absorbing products which are pigmented and/or otherwise colored so as to supplement or correspond to the desired camouflage pattern and colors thereof.

### SUMMARY OF THE INVENTION

The invention generally provides methods of, and corresponding compositions for, generally simultaneously camouflaging and suppressing odors by applying at least one odor absorbing agent directly to a user and/or to hunting garments and/or to hunting accessories. The odor absorbing agents are applied to such user and/or to hunting garments and/or hunting accessories in a manner which facilitates a reduction in the relative visual conspicuousness of the user.

In a first family of embodiments, the invention comprehends a method of suppressing odor and visually camouflaging, the method comprising applying a dry powder form of an odor suppressing agent to hunting garments; and applying a dry powder form of an odor suppressing agent to exposed skin surfaces of a user.

In some embodiments, the odor suppressing agent is applied by dispensing from a bottle.

In some embodiments, such dry powder form of the odor suppressing agent is applied to hunting garments by sealing the garments in a bag containing the agent and agitating the contents of the bag.

In some embodiments, the method further includes applying a plurality of relatively differently colored odor suppressing agents to hunting garments.

In some embodiments, the hunting garments define a camouflage pattern.

In some embodiments, the hunting garments define a camouflage pattern and the differently colored odor suppressing agents are applied to the garments in a manner that visually corresponds to the camouflage pattern.

In a second family of embodiments, the invention comprehends a method of generally simultaneously camouflaging and suppressing odor, the method comprising applying an odor suppressing agent, carried in a liquid carrier, to hunting garments; and applying a dry powder form of an odor suppressing agent to exposed skin surfaces of a user.

In some embodiments, the liquid carrier is an aqueous solution.

In some embodiments, the method further includes forming a solution within a container, the solution including the odor suppressing agent, thence placing the hunting garments into the solution and agitating the garments therein.

In some embodiments, the method further includes forming a solution within a container, by admixing an odor suppressing tablet and a volume of liquid together, thence placing the hunting garments into the solution and agitating the garments therein.

In a third family of embodiments, the invention comprehends a method of generally simultaneously suppressing odor and visually camouflaging, the method comprising applying a first odor absorbing agent to an exposed skin surface of a user; and applying a second odor absorbing agent to an exposed skin surface of the user, wherein the first and second odor absorbing agents define first and second colors which differ from each other.

In some embodiments, the first color defines a green color and the second color defines a brown color.

In some embodiments, the first color defines a green color and the second color defines a black color.

In some embodiments, the first color defines a relatively lighter color and the second color defines a relatively darker color.

In some embodiments, ones of the first and second odor absorbing agents are applied with a brush.

In a fourth family of embodiments, the invention comprehends a kit for generally simultaneously camouflaging and suppressing odor, the kit comprising a first odor absorbing agent which defines a first color; and a second odor absorbing agent which defines a second, different, color; and at least one delivery device adapted and configured to deliver ones of the first and second odor absorbing agents from such device to a user.

In some embodiments, the delivery device comprises at least one bottle.

In some embodiments, the delivery device comprises a compressed form of at least one of the first and second odor absorbing agents.

In some embodiments, the compressed form of at the least one of the first and second odor absorbing agents generally defines a tablet configuration.

In some embodiments, at least one of the first and second odor absorbing agents is housed in a container and defines a predetermined volume of the agent.

### BRIEF DESCRIPTION OF THE DRAWINGS

FIG. 1A illustrates a pictorial view of a first embodiment of odor absorbing substance delivery devices of the invention, holding an odor absorbing agent of a first color therein.

FIG. 1B illustrates a pictorial view of a first embodiment of odor absorbing substance delivery devices of the invention, holding an odor absorbing agent of a second color therein.

FIG. 1C illustrates a pictorial view of a first embodiment of odor absorbing substance delivery devices of the invention, holding an odor absorbing agent of a third color therein.

FIG. 2A illustrates a pictorial view of a second embodiment of odor absorbing substance delivery devices of the invention, holding an odor absorbing agent of a first color therein.

FIG. 2B illustrates a pictorial view of a second embodiment of odor absorbing substance delivery devices of the invention, holding an odor absorbing agent of a second color therein.

FIG. 2C illustrates a pictorial view of a second embodiment of odor absorbing substance delivery devices of the invention, holding an odor absorbing agent of a third color therein.

FIG. 3A illustrates pictorial views of a third embodiment of odor absorbing substance delivery devices, of a first color, of the invention.

FIG. 3B illustrates pictorial views of a third embodiment of odor absorbing substance delivery devices, of a second color, of the invention.

FIG. 3C illustrates pictorial views of a third embodiment of odor absorbing substance delivery devices, of a third color, of the invention.

FIG. 3D illustrates a pictorial view of a variant of the odor absorbing delivery device of FIG. 3C.

FIG. 4 illustrates a pictorial view of a fourth embodiment of odor absorbing substance delivery devices of the invention.

FIG. 5 illustrates a front elevation view of the odor absorbing substance delivery device of FIG. 4, in use.

FIG. 6A illustrates a pictorial view of a fifth embodiment of odor absorbing substance delivery devices of the invention.

FIG. 6B illustrates a pictorial view of a variant of the odor absorbing substance delivery device of FIG. 6A.

FIG. 6C illustrates a pictorial view of another variant of the odor absorbing substance delivery device of FIG. 6A.

FIG. 7 illustrates a pictorial view of the odor absorbing substance delivery devices of FIG. 6A-6C, in use.

FIG. 8 illustrates a pictorial view of a sixth embodiment of odor absorbing substance delivery devices of the invention.

FIG. 9 illustrates a pictorial view of the odor absorbing substance delivery device of FIG. 8, in use.

FIG. 10 illustrates a pictorial view of a seventh embodiment of odor absorbing substance delivery devices of the invention.

FIG. 11 illustrates a pictorial view of the odor absorbing substance delivery device of FIG. 10, in use.

FIG. 12 illustrates a pictorial view of a first embodiment of odor absorbing garments.

FIG. 13A illustrates an exploded view of the odor absorbing garment of FIG. 1.

FIG. 13B illustrates a pictorial view of a second embodiment of odor absorbing garments.

FIG. 14A illustrates a top plan view of a first disposable odor absorbing article, in a generally air tight enclosure.

FIG. 14B illustrates a top plan view of a second disposable odor absorbing article, in a generally air tight enclosure.

### DETAILED DESCRIPTION OF THE ILLUSTRATED EMBODIMENTS

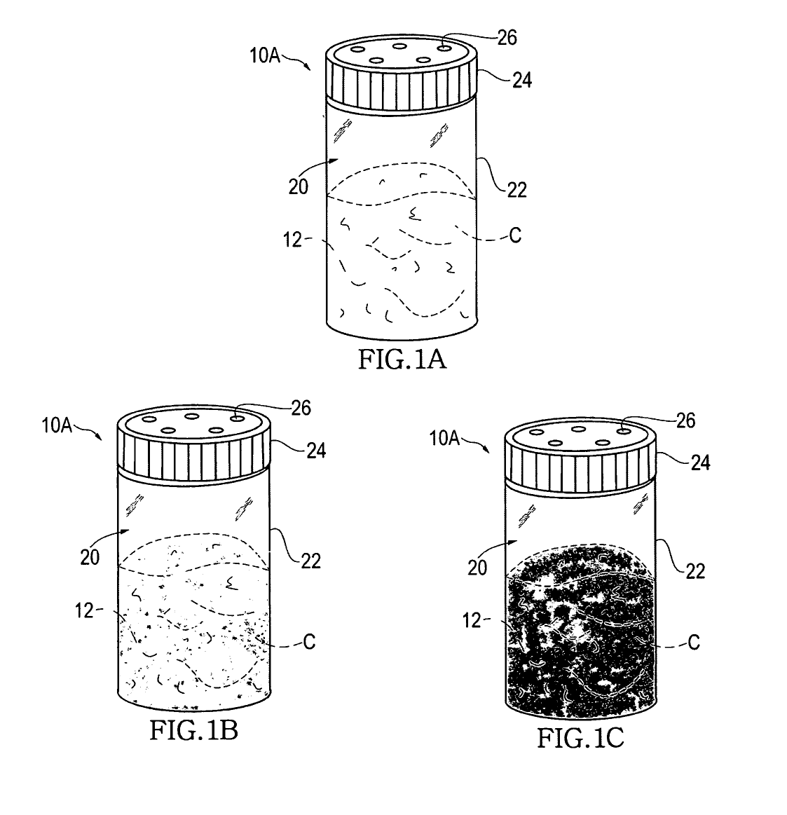

Referring generally to FIGS. 1A, 1B, 1C, 2A, 2B, 2C, 3A, 3B, and 3C, in typical implementations, the invention comprehends an odor absorbing substance, deodorizer or other odor masking agent, e.g. odor absorbing agent 12.

Exemplary suitable odor absorbing substances, deodorizers or other odor masking agents include, but are not limited to, various well known and commercially available products which contain, for example ones of activated charcoal, zeolite, colloidal silver, silver ions, sodium bicarbonate, triclosan, hydrogen peroxide, various surfactants such as iron chloride, calcium nitrates and others, as well as other odor absorbing substances, deodorizers and/or other odor masking agents such as various silicon-based nanophase materials e.g. aerogels or aerogel containing materials, and others.

Specifically regarding various carbon based embodiments of odor absorbing agent 12, the agent is any of a variety of suitable carbon powders, and preferably activated forms of carbon. Suitable varieties of activated carbon include, but are not limited to, coal activated carbon, lignite activated carbon, wood activated carbon, coconut activated carbon, and/or others.

In some embodiments, odor absorbing agent 12 retains its typical color, hue, and other visual or appearance characteristics, whilst in other embodiments the color, hue, and other visual or appearance characteristics of odor absorbing agent 12 are modified, as desired. Accordingly, generally as used herein, the phrase “odor absorbing agent 12” and variants thereof includes embodiments of both color modified and non-color modified odor absorbing agents.

Specifically regarding color-modified embodiments of odor absorbing agent 12, the agent is e.g. dyed, stained, pigmented, and/or otherwise colored, by way of e.g. colorant “C,” or otherwise as desired, which enables a user to, for example, apply such odor absorbing product as both (i) an odor absorbing product, and (ii) a camouflage and/or other visual masking product.

In other words, in some embodiments, odor absorbing agent 12 is colored and configured, as desired, to correspond to, complement, supplement, or enable the user to utilize the odor absorbing agent as, a camouflage pattern, scheme, or other indicia. Accordingly, in some embodiments, odor absorbing agent 12 is a multifunctional, odor suppression or masking, and visually concealing or camouflaging product, adapted and configured for initial application and/or reapplication to various substrates.

In other embodiments, odor absorbing agents 12 are not colored, beyond any coloring or bleaching, or other pigmentation application or pigmentation/color removal which is considered typical for such odor absorbing agents. Rather, some odor absorbing agents 12 retain their usual, commercially available, color and appearance. As one example, in some embodiments, odor absorbing agent 12 is e.g. carbon powder and is generally dark gray or black in color. As another example, in some embodiments, odor absorbing agent 12 is e.g. zeolite or baking soda and is generally white or a shade of off-white in color.

In yet other embodiments, the color of odor absorbing agent 12 is the result of modification to the odor absorbing agent, as desired, to result in a relatively differently colored or otherwise visually modified odor absorbing agent. This is done by way of, for example, colorant “C” which enables e.g. additive colorant methods or subtractive colorant methods.

In additive colorant methods, colorant “C” is added to and e.g. reacts or otherwise interfaces with odor absorbing agent 12. In such embodiments, colorant “C” is a dye, paint, ink, pigment, and/or other colorant source, which imparts at least some color-based qualities upon odor absorbing agent 12.

Optionally, colorant “C” is, itself, associated with a carrier substance. Thus, colorant “C” can be an emulsion or otherwise suspended or disbursed in, e.g. a paste, grease, lotion, oil, cream, or various waxes such as cetyl palmitate, triacontanyl palmitate, octadecyl octadecanoate or stearyl stearate, dodecyl hexadecanoate, carnauba, lanolin, glycol disterate, and/or others, as a carrier or base. Thence, odor absorbing agent 12 is added to such colored emulsion to provide the color-modified odor absorbing agent 12.

In other additive colorant methods, the particles of colorant “C” remain physically and/or chemically distinct from the odor absorbing agent 12 particles, yet are, for example, admixed therewith. The resultant mixture is e.g. a dispersion, suspension, slurry, blend, interminglement and/or other combination of colored particles and odor absorbing particles which has an overall color different from the color of the odor absorbing agent 12 particles, alone.

In subtractive colorant methods, the colorant “C” removes or modifies some of the, for example, color pigment from odor absorbing agent 12 by way of bleaching, lightening or otherwise.

The various shades, hues, and/or other color characteristics, of colorant “C” are selected so as to provide the desired color or visual appearance to odor absorbing agent 12. By suitably selecting the appropriate shades, hues, other color and visual characteristics, and determining the relative concentrations of each of colorant “C” and odor absorbing agent 12, the user is able to impart the desired color quality and characteristics to odor absorbing agent 12.

In other words, by selecting the color, volume, and concentration, of colorant “C” for use with odor absorbing agent 12, the user creates any desired color along the visible light spectrum i.e. within the range of colors between red, orange, yellow, green, blue, and violet.

For embodiments of odor absorbing agent 12 which are also used as camouflage and/or to compliment or supplement other camouflage, the most commonly utilized and preferred colors of odor absorbing agent 12 are white, tan, yellow, grey, green, blue, red, brown, black, and others, as well as the various shades, hues, and/or other variants and combinations thereof.

Referring now to various substrates to which odor absorbing agent 12 is applied, exemplary suitable substrates include, but are not limited to, e.g. garments and/or clothing articles, such as e.g. undergarments, socks, shoes, boots, pants, trousers, coveralls, bibs, shirts, sweaters, sweatshirts, coats, gloves, mittens, hats, caps, masks, and others, as well as various portions thereof.

Other suitable application substrates and/or articles include, but are not limited to, e.g. various non-personal and non-clothing articles such as e.g. pieces of cloth or other flexible hunting-blind and/or wind blocking materials e.g. various woven, non-woven, mesh, knitted, netting, cloth, fabric, textile, and/or other material including, but not limited to, canvas, naugahyde, burlap, tarp material, and/or other suitable natural, synthetic, semi-synthetic cloths, fabrics, and/or other generally flexible, resilient and non-resilient materials.

Yet other suitable application substrates and/or articles include, but are not limited to various other game hunting or game observing accessories, e.g. gear bags, all terrain vehicle (ATV) saddlebags, ATV tank-bags, various other ATV cargo holding devices and carrying devices, and others.

In addition to the abovementioned inanimate substrates and articles, various ones of the odor absorbing agents and methods of applying disclosed elsewhere are, in some instances, equally applicable for application directly to the body of the user e.g. human epidermis, namely various exposed or unexposed skin surfaces of a user, such as the face, arms, underarms, hands, feet, and others, and other suitable substrates such as e.g. the hair of a user, and others, and are thus also well within the scope of the invention.

The user applies and/or reapplies odor absorbing agent 12 to ones of such substrates by way of various suitable delivery devices and methods. FIGS. 1A, 1B, 1C show a pictorial view of a first embodiment of odor absorbing substance delivery devices of the invention.

The various odor absorbing substance delivery devices enable a user to e.g. recharge the odor absorbing characteristics, effectiveness, and functionality of the substrate such as garment “G” (FIGS. 4, 5, 7, 12, 13A, and 13B) by applying or reapplying odor absorbing agent 12 thereto.

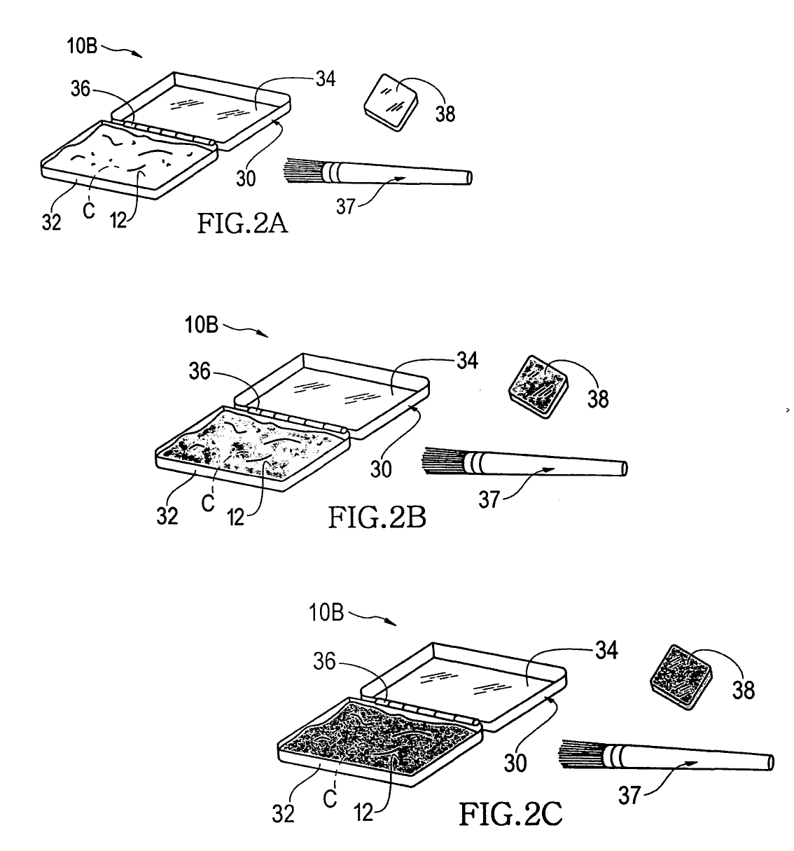

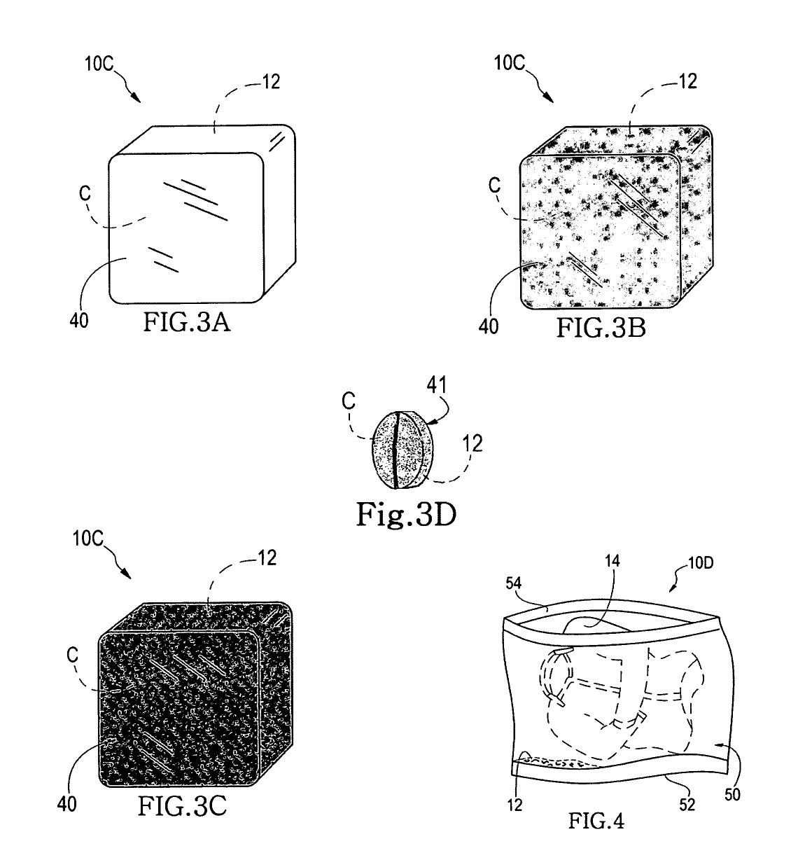

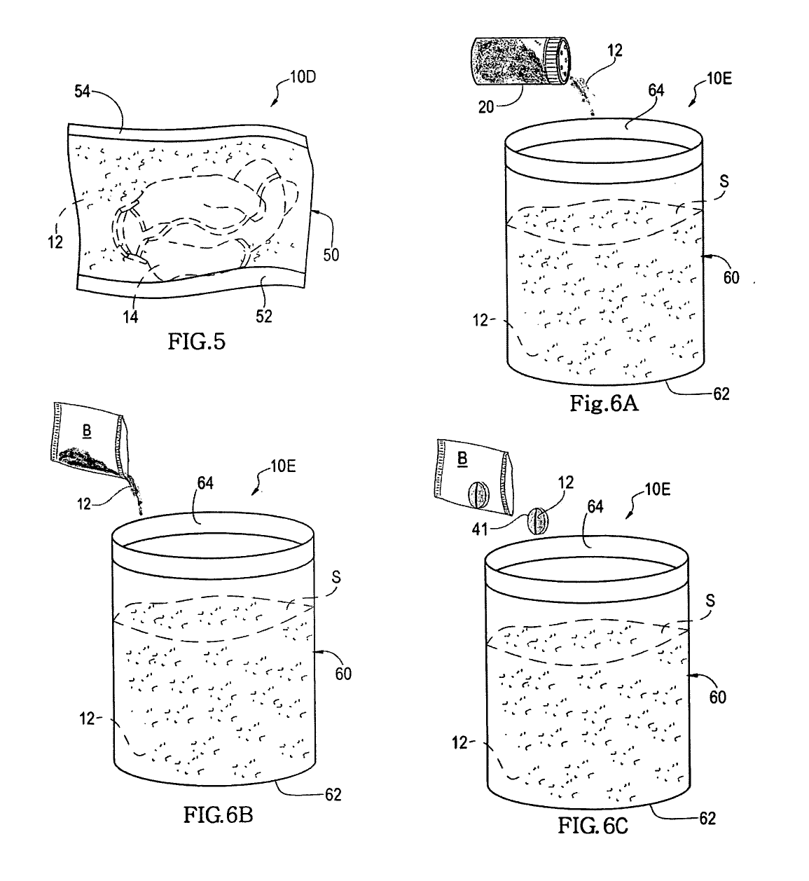

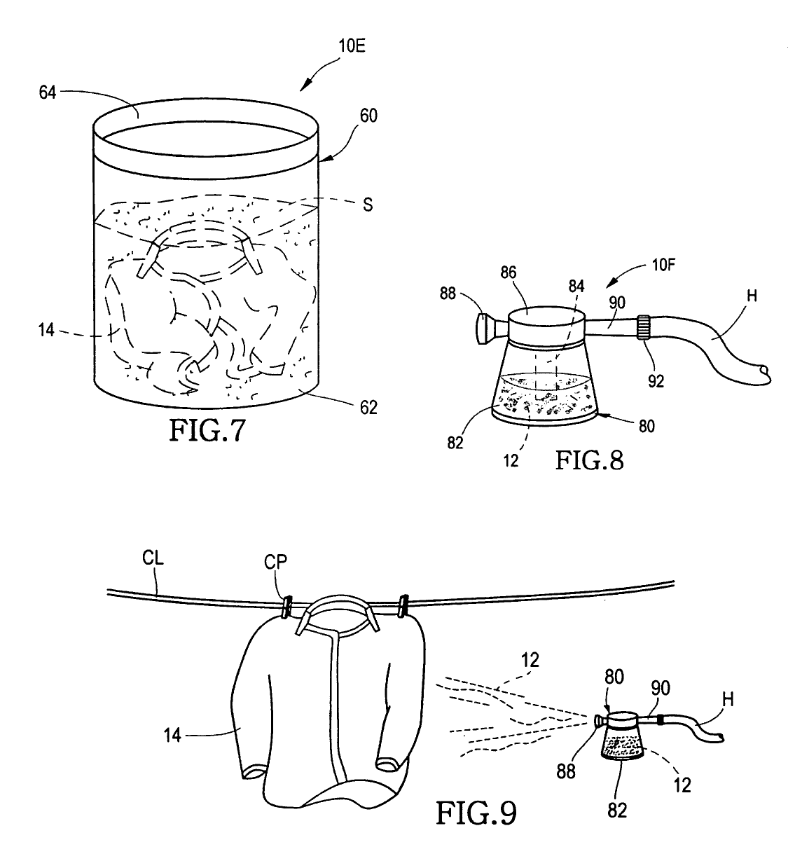

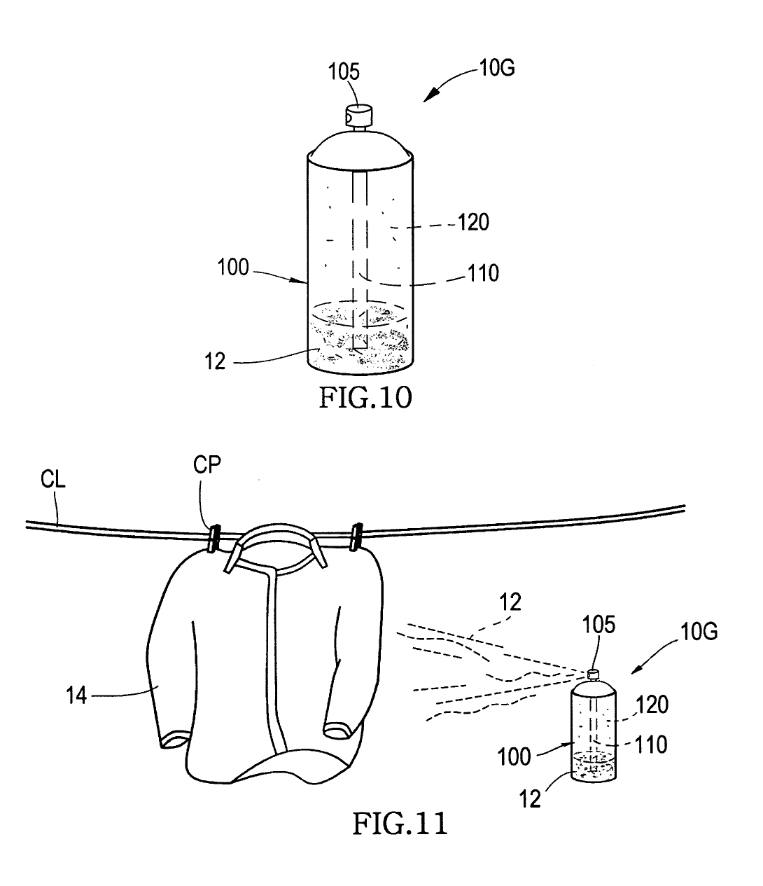

In a typical implementation of the invention, an odor absorbing agent delivery device 10A (FIGS. 1A, 1B, 1C), 10B (FIGS. 2A, 2B, 2C), 10C (FIGS. 3A, 3B, 3C), 10D (FIGS. 4 and 5), 10E (FIGS. 6A, 6B, and 7), 10F (FIGS. 8 and 9), 10G (FIGS. 10 and 11), includes an odor absorbing agent 12, and a means of applying odor absorbing agent 12 to a suitable substrate.

Referring now to FIGS. 1A, 1B, 1C, odor absorbing substance delivery device 10A includes odor absorbing agent 12, and bottle 20. Bottle 20 includes bottle-body 22 and lid 24.

Bottle-body 22 is a generally cylindrical member with a generally circular bottom wall and a circumferential outer wall which extends upwardly from the upper surface of the bottom wall. The inwardly facing surface of the outer circumferential sidewall generally defines the outer perimeter of a cavity, which extends from the upper portion of the bottle-body 22, downwardly thereinto.

Lid 24 has a generally circular upper surface and an outer circumferential sidewall. A portion of the sidewall of lid 24 has threads which extend inwardly from an inner surface thereof. An upper and outer portion of the sidewall of bottle-body 22 has cooperating threads which extend outwardly therefrom. In other words, lid 24 is adapted and configured to threadedly and removably attach to bottle-body 22, so as to provide a generally enclosed structure which houses the odor absorbing agent 12.

Ones of a plurality of apertures, namely apertures 26, extend axially through the entire thickness dimension of the upper surface of lid 24. Accordingly, as desired, a user can sprinkle, shake, and/or otherwise dispense odor absorbing agent 12 from bottle 20. The particular size, shape, spacing between, and other characteristics of ones of apertures 26 are selected to provide the desired user characteristics of bottle 20.

Accordingly, when the user desires to dispense odor absorbing agent 12 at a relatively more rapid rate or in relatively high volumes, apertures 26 are relatively larger. And when the user desires to dispense odor absorbing agent 12 at a relatively less rapid rate or in relatively lower volumes, apertures 26 are relatively smaller.

In some embodiments, apertures 26 generally define variable diameter openings. As one example, lid 24 is rotatable, whereby rotation of lid 24 in a first direction generally increases the size of apertures 26 until a maximum aperture size is realized, i.e. the apertures are fully open, and rotation of lid 24 in a second, opposite, direction generally decreases the size of apertures 26 until a minimum aperture size is realized, i.e. the apertures are fully closed.

The various odor absorbing agents 12 of FIGS. 1A, 1B, and 1C, illustrate odor absorbing agents which are combined with different colors, different concentrations, and/or or different volumes of colorant “C”. Accordingly, as illustrated, odor absorbing agent 12 of FIG. 1A is a first color. Odor absorbing agent 12 of FIG. 1B is a second, relatively darker, color. Odor absorbing agent 12 of FIG. 1C is a third color which is relatively darker than the odor absorbing agents of FIGS. 1A and 1B.

Referring now to FIGS. 2A, 2B, 2C, odor absorbing substance delivery device 10B includes odor absorbing agent 12, box container 30, optionally ones of brush 37 and sponge 38. Box container 30 includes container base 32, container cover 34, and hinge 36.

Container base 32 is a generally shallow, concave tray. Namely, container base 32 includes a bottom wall and a plurality of sidewalls which extend upwardly from the outer perimeter of the bottom wall. Respective ends of the sidewalls are connected to the corresponding other ends of the sidewalls. The concavity of container base 32 generally houses odor absorbing agent 12.

Container cover 34 is a generally shallow, concave tray, e.g. similar to container base 32. Container cover 34 includes a top wall and a plurality of sidewalls which extend downwardly from the outer perimeter of the bottom wall, when the container 30 is in the closed configuration. Respective ends of the sidewalls are connected to the corresponding other ends of the sidewalls.

Hinge 36 hingedly connects container base 32 and container cover 34. In other words, hinge 36 enables box container 30 to pivotably open and/or pivotably close, by way of the hinged relationship between container base 32 and container cover 34.

Brush 37 includes a generally rigid handle portion and at least one bristled end portion. The bristles of the brush bristled end portion are adapted and configured to transfer, as desired by the user, some of the odor absorbing agent 12 thereupon, and subsequently deposit some of the odor absorbing agent 12 upon the application substrate, be it skin, hair, clothing, garments, hunting-blinds, or otherwise.

Sponge 38 is a resiliently flexible member. Sponge 38 is adapted and configured to transfer, as desired by the user, some of the odor absorbing agent 12 thereupon, and subsequently deposit some of the odor absorbing agent 12 upon the application substrate, be it skin, hair, clothing, garments, hunting-blinds, or otherwise. Sponge 38 can be any of a variety of suitable commercially available sponges, including, but not limited to, latex foam sponges, non-latex based sponges, hydra sponges, nylon stipple sponges, and/or others.

The various odor absorbing agents 12 of FIGS. 2A, 2B, and 2C, illustrate odor absorbing agents which are combined with different colors, different concentrations, and/or or different volumes of colorant “C”. Accordingly, as illustrated, odor absorbing agent 12 of FIG. 2A is a first color. Odor absorbing agent 12 of FIG. 2B is a second, relatively darker, color. Odor absorbing agent 12 of FIG. 2C is a third color which is relatively darker than the odor absorbing agents of FIGS. 2A and 2B.

Optionally, an individual one of odor absorbing substance delivery devices 10A, 10B, or others, includes a plurality of colors of odor absorbing agent 12. As one example, ones of the odor absorbing substance delivery devices, for example delivery device 10A or 10B (or others) can include a plurality of distinct compartments therein, e.g. compartments which are separated by longitudinally extending separation walls, whereby a single odor absorbing substance delivery device 10A or 10B is adapted and configured to house and deliver a plurality of differently colored odor absorbing agents 12 therefrom. Accordingly, an individual delivery device 10A, 10B, and/or others, can define a skin camouflaging or other substrate camouflaging kit, having a plurality of differently colored odor absorbing agents 12 therein.

Referring now to FIGS. 3A, 3B, 3C, odor absorbing substance delivery device 10C is largely the odor absorbing substance, itself. In other words, the odor absorbing substance is it's own applicator. As one example, particles of the odor absorbing agent 12 are compressed, under sufficiently high pressure, whereby the result is a compressed block, brick, sphere, or any desired shaped, which can be handled by the user and sufficiently resists crumbling during use, e.g. block 40. As desired, in addition to odor absorbing agent 12, odor absorbing substance delivery device 10C, namely block 40, includes various other components to enhance the affinity of the particles to each other, or which otherwise improve the structural integrity of the compressed block, e.g. various suitable binders, adhesives, and/or others.

Like those of FIGS. 1A-2C, the various odor absorbing agents 12 of FIGS. 3A, 3B, and 3C, illustrate odor absorbing agents which are combined with different colors, different concentrations, and/or or different volumes of colorant “C”. Accordingly, as illustrated, odor absorbing agent 12 of FIG. 3A is a first color. Odor absorbing agent 12 of FIG. 3B is a second, relatively darker, color. Odor absorbing agent 12 of FIG. 3C is a third color which is relatively darker than the odor absorbing agents of FIGS. 3A and 3B.

Referring now FIG. 3D, the odor absorbing substance delivery device is again largely the odor absorbing substance, itself. However, in this embodiment, it is relatively less suited for direct-pressure application, as compared to block 40. Rather it is adapted and configured to e.g. dissolve in a liquid carrier and be applied to the substrate therewith (FIG. 6C), which is described in greater detail elsewhere herein. In other words, the odor absorbing agent 12 is formed into odor absorbing tablets 41.

Tablets 41, like block 40, include various other components to enhance or suitably maintain the structural integrity of the tablet, e.g. various suitable binders, adhesives, and/or others. However, tablet 41 further includes various solubility aids, preferably those which enhance or suitably maintain strength and durability of the tablet 41, to resist damage and/or breakage during storage and transportation. Those skilled in the art are well aware of suitable methods of tablet formation and manufacture, which are suitable for making tablets 41. Those methods include compacting the constituent particles and compacting pressures and, as required, adding various suitable binders to provide a cohesive-like effect.

Referring now to FIG. 4, odor absorbing substance delivery device 10D includes odor absorbing agent 12 and flexible enclosure 50. Flexible enclosure 50 is e.g. a bag, or other enclosure which is generally flexible, and is generally adapted and configured to apply an odor absorbing substance to inanimate objects such as clothing and/or other garments, flexible hunting-blind materials, wind-blocking materials, and/or others.

Flexible enclosure 50 includes lower portion 52 and upper portion 54. Lower portion 52 is generally closed or sealed. Upper portion 54 is generally open, yet selectably closable or sealable, as desired by a user. The selectably closable feature of flexible enclosure 50 enables a user to generally restrain, confine, and keep, odor absorbing agent 12 inside of the flexible enclosure 50 while treating the object(s) or article(s) therein, imparting, at least temporarily, odor absorbing characteristics thereto.

Referring now to FIGS. 6A, 6B, and 6C, odor absorbing substance delivery device 10E includes odor absorbing agent 12, container 60, and solvent “S.” Solvent “S” is a liquid, preferably water, which can suspend odor absorbing agent 12 therein, dissolve odor absorbing agent 12 therein, and/or otherwise carry odor absorbing agent 12 therein.

As exemplarily illustrated, container 60 is a generally cylindrical member with a generally circular bottom wall and a circumferential outer wall which extends upwardly from the upper surface of the bottom wall, although in some embodiments, as with the containers of FIGS. 1A-1C, the bottom wall is polygonal and the container includes a plurality of sidewalls.

Container 60 includes lower portion 62 and upper portion 64, and is adapted and configured to suitably hold liquid e.g. solvent “S” therein. The inwardly facing surface of the outer circumferential sidewall generally defines the outer perimeter of a cavity, which extends from upper portion 64 downwardly into container 60, toward lower portion 62. Suitable containers for use as container 60 include a variety of e.g. buckets, basins, barrels, and/or other containers.

Referring now to FIG. 8, odor absorbing substance delivery device 10F includes hose “H,” odor absorbing agent 12, and hose-end sprayer 80. Hose-end sprayer 80 includes jar 82, spray mechanism 84, jar-cover 86, nozzle 88, handle 90, and coupling mechanism 92. Hose “H” is a conventional garden or utility hose which is connected at a first end to a pressurized source of water and at a second end to hose-end sprayer 80.

Jar 82, as illustrated, is a generally tapering cylindrical member with a generally circular bottom wall and a circumferential outer wall which extends upwardly, and taperingly, from the upper surface of the bottom wall. Jar 82 is adapted and configured to temporarily house odor absorbing agent 12 therein, and provide a structure in which odor absorbing agent 12 mixes with, suspends in, optionally dissolves in, e.g. water, which is ultimately applied to an application substrate.

Jar-cover 86 is e.g. threadedly attached to the upper portion of jar 82. Jar-cover 86 provides mounting structure by which various other components of hose-end sprayer 80, e.g. spray mechanism 84, nozzle 88, handle 90, optionally others, are attached to the remainder of hose-end sprayer 80.

Spray mechanism 84 communicates with the input and output portions of hose-end sprayer 80 and portions thereof extend downwardly, from the lower surface of jar-cover 86, into the cavity of jar 82. As desired, spray mechanism 84 includes a regulator which enables a user to adjust the mix rate of the hose-end sprayer 80, optionally the volume of liquid which flows from hose-end sprayer 80, optionally an anti-siphon device to prevent or mitigate the amount of contents of jar 82 which can siphon back into hose “H.”

Nozzle 88 is mounted to and extends from an outer surface of jar-cover 86, and faces a first direction. Nozzle 88 is adapted and configured to spray or mist a stream or fog of liquid therethrough, such as through one or more apertures or orifices. Namely, the contents of jar 82 pass through portions of spray mechanism 84, and ultimately exit hose-end sprayer 80 by way of, e.g. through, nozzle 88.

Handle 90 is an elongate, generally rigid, member with first and second ends, and is adapted and configured to enable a user to grip and manipulate the hose-end sprayer 80, by way of such handle. The first end of handle 90 is attached to an outer surface of jar-cover 86. The second end of handle 90 is distal jar-cover 86 and proximate hose “H.” A bore extends axially through handle 90, and communicates with hose “H” at a first end thereof and with spray mechanism 84 at a second end thereof. In other words, handle 90 serves as a conduit through which water, emitted from hose “H,” passes and handle 90 routes such water into and through various portions of the remainder of hose-end sprayer 80.

Coupling mechanism 92 is attached to the second end of handle 90, namely the end which is distal jar-cover 86. Coupling mechanism 92 has a threaded inner circumferential surface which is adapted and configured to cooperate with the threaded outer circumferential surface at the end of hose “H.” Accordingly, hose “H” is attached to hose-end sprayer 80 through the mechanically interfacing relationship between hose “H” and the hose-end sprayer.

Exemplary of a suitable hose-end sprayer is Gilmour Model Number 499 hose-end sprayer, available from Gilmour® Gardening Innovation, Somerset, Pa.

Referring now to FIG. 10, odor absorbing substance delivery device 10G includes odor absorbing agent 12, contained in and delivered by way of aerosol can 100. Aerosol can 100 includes a generally cylindrical body, nozzle 105, delivery tube 110, and propellant 120.

Nozzle 105 communicates with the upper end of delivery tube 100. A suitable seal and/or valve mechanism, e.g. a valve/seal mechanism, provides a cooperative interface between the upper end of delivery tube 100 and nozzle 105. The lower end of delivery tube 100 operatively extends downwardly into the product to be delivered, namely odor absorbing agent 12.

The contents of aerosol can 100 are pressurized. Namely, a sufficient volume of propellant 120 is provided to the cavity of the cylindrical body to suitably pressurize aerosol can 100. Propellant 120 is a fluid which has a boiling point below that of room temperature. Exemplary of fluids with such qualities and characteristics, suitable for use as propellant 120 include, but are not limited to, various liquefied petroleum gas (LPG), Di Methyl Ether, Chlorofluorocarbons (CFC's), various of the non-soluble compressed gasses such as compressed ambient air and nitrogen, various of the soluble compressed gasses such as carbon dioxide, and/or others.

When a user depresses nozzle 105, the valve of the valve/seal mechanism opens whereby a passage is created between the inside of the can and the outside of the can, through the nozzle. Under this condition, propellant 120, which is pressurized, drives the odor absorbing agent 12 outwardly though delivery tube 100 and nozzle 105.

As desired, multiple, different, colors of odor absorbing agents 12 are contained in multiple, separate ones of odor absorbing substance delivery devices 10A, 10B, 10C, 10D, 10E, 10F and 10G. Ones of the multiple odor absorbing substance delivery devices 10A, 10B, 10C, 10D, 10E, 10F and 10G, which contained distinctly colored odor absorbing agents 12, can be packaged together to define a single camouflaging and odor absorbing kit.

To use the various odor absorbing substance delivery devices 10A, 10B, 10C, 10D, 10E, 10F and 10G, the user first selects the desired application substrate or substrates. Then the user evaluates and determines the amount of odor absorbing agent 12 to be applied thereto. Such evaluation and determination is based, at least in part, on various factors which correspond to the respective state of a particular application substrate.

Also, depending on, for example, the particular game animal which is hunted or observed, a user can apply relatively less or more odor absorbing agent 12 to the particular substrate. Accordingly, as one example, a user can apply a relatively greater volume of odor absorbing agent 12 directly to the user's skin when hunting game with a relatively good sense of smell and a relatively lesser volume of odor absorbing agent 12 when hunting game with a relatively poor sense of smell.

As another example, when a user desires to apply odor absorbing agent 12 to the user's hunting garments, the user can apply a relatively lesser volume of odor absorbing agent 12 if the garments are relatively new, clean, and generally free of odors. Correspondingly, also when a user desires to apply odor absorbing agent 12 to the user's hunting garments, the user can apply a relatively greater volume of odor absorbing agent 12 if such garments are relatively old, dirty or otherwise soiled, and generally odorous.

As another example, when a user desires to apply odor absorbing agent 12 to hunting blind material or wind-blocking material, the user can apply a relatively lesser volume of odor absorbing agent 12 if the materials are relatively new, clean, and generally free of odors. Correspondingly, when a user desires to apply odor absorbing agent 12 to such materials, the user can apply a relatively greater volume of odor absorbing agent 12 if such materials are relatively old, dirty or otherwise soiled, and generally odorous.

In some embodiments, the odor absorbing agents 12 are applied as a generally dry powder or other dry substance. As one example, referring to FIGS. 1A, 1B, 1C, as desired, a user uses odor absorbing substance delivery device 10A to apply odor absorbing agent 12 to the user's skin and hunting garments. First, the user shakes, sprinkles, pours, and/or otherwise dispenses odor absorbing agent 12 from bottle 20 into the palm(s) of the user's hand(s). Then, the user smears, wipes, rubs-in, and/or otherwise applies the odor absorbing agent 12 onto desired exposed skin surfaces.

Then, as desired, the user applies odor absorbing agent 12 to the user's hunting garments. Namely, the user first shakes, sprinkles, pours, and/or otherwise dispenses odor absorbing agent 12 from bottle 20 into the palm(s) of the user's hand(s). Next, the user smears, wipes, rubs-in, and/or otherwise applies the odor absorbing agent 12 onto desired hunting garments.

When applying, for example, multiple colors of odor absorbing agent 12 directly to the user's skin, the user can apply the various colors in a camouflage type pattern, as desired. In so doing, the user enjoys both the odor absorbing quality of agent 12 and its camouflaging functionality. Correspondingly, when used with and applied directly to hunting garments, the user can apply the various different colors of odor absorbing agents 12 to correspond with the e.g. camouflage pattern of the garment.

To remove the odor absorbing agent 12 from the skin of the user, the user washes it therefrom using soap and water. The process of applying odor absorbing agent 12 to the user's skin, clothing/garments, and/or other hunting accessories is preferably performed before each hunting expedition, or observation or photography session.

Referring now to FIGS. 2A, 2B, 2C, as desired, a user uses odor absorbing substance delivery device 10B to apply odor absorbing agent 12 to the user's skin and hunting garments. First, the user applies odor absorbing agent 12 to brush 37 and/or sponge 38. Then, the user transfers the odor absorbing agent 12 from brush 37 and/or sponge 38 to desired exposed skin surfaces. Then, as desired, the user smears, wipes, rubs-in, and/or otherwise applies the odor absorbing agent 12, with e.g. the user's hand(s), onto such skin surfaces.

Then, as desired, the user applies odor absorbing agent 12 to the user's hunting garments. Namely, the user first applies odor absorbing agent 12 to brush 37 and/or sponge 38. Then, the user transfers the odor absorbing agent 12 from brush 37 and/or sponge 38 to the desired hunting garments and/or hunting accessories. Then, as desired, the user smears, wipes, rubs-in, and/or otherwise applies the odor absorbing agent 12, with e.g. the user's hand(s), onto such hunting garments and/or hunting accessories.

Referring now to FIGS. 3A, 3B, 3C, as desired, a user uses odor absorbing substance delivery device 10C to apply odor absorbing agent 12 to the user's skin, hunting garments, and/or other hunting accessories. Namely, the user grasps block 40 and rubs, smears, and/or otherwise applies, the odor absorbing agent 12 to the user's skin, hunting garments, and/or other hunting accessories.

Referring now to FIG. 4, as desired, a user uses odor absorbing substance delivery device 10C to apply odor absorbing agent 12 to the garment “G” and/or other hunting accessories. First, the user pours or dispenses the desired amount of odor absorbing agent 12 into bag 50. Then, the user inserts the garment “G” and/or other hunting accessories into bag 50, and closes and seals the bag. Referring now to FIG. 5, the user then shakes bag 50 so as to cause at least some of the odor absorbing agent 12 to become airborne, and to jostle or agitate the other contents of the bag e.g. the garment “G” and/or other hunting accessories. The agitation of the bag contents generally disburses odor absorbing agent 12 onto the surfaces of the garment “G” and/or other hunting accessories.

In some embodiments, the odor absorbing agents 12 are applied in a generally wet condition, or by way of a liquid carrier. As one example, referring to FIGS. 6A, 6B, 6C, and 7, as desired, a user uses odor absorbing substance delivery device 10E to apply odor absorbing agent 12 to e.g. garment “G” and/or other hunting accessories by way of a mixture suspending the odor absorbing agent 12 therein.

First, a user dispenses a volume of solvent “S,” which is preferably water but can be other suitable solvents, and a volume of odor absorbing agent 12, into container 60. Exemplary suitable ratios of odor absorbing agent volume to solvent “S” volume is about 1 tablespoon of odor absorbing agent per about 5 gallons of solvent “S,” optionally about 3 tablespoons of odor absorbing agent per about 4 gallons of solvent “S,” optionally about 5 tablespoons of odor absorbing agent per about 3 gallons of solvent “S,” optionally about 6 tablespoons of odor absorbing agent per about 2 gallons of solvent “S,” optionally others.

The odor absorbing agent 12 is dispensed into solvent “S” in any of a variety of suitable ways. As one example, odor absorbing agent 12 is dispensed directly from bottle 20 into the solvent “S” (FIG. 6A).

As another example, a prepackaged and predetermined amount of odor absorbing agent 12 is sealed in or otherwise contained in a pouch, bag, or wrapper, e.g. bag “B” (FIG. 6B). Bag “B” is preferably made from a polymeric film with low gas transmissibility characteristics and optionally is a multilayer film which includes an oxygen barrier layer therein. In such embodiments, the user merely opens bag “B” and pours its entire contents into solvent “S”.

As yet another embodiment, as in FIG. 6C, bag “B” contains at least one tablet 41 therein. Upon entering solvent “S”, tablet 41 dissolves, dissociates, disunites, and/or otherwise breaks up, therein. Here again, the user merely opens bag “B” and expels its contents, tablet(s) 41, into solvent “S.”

Once the odor absorbing agent solution, slurry, or mixture is prepared, by way of stirring, agitating, or otherwise, the user submerges the garment “G” and/or other hunting accessory into the solution contained in container 60. The user then stirs, washes, rinses, and/or otherwise agitates, the garments “G” and/or other hunting accessories therein. The user then hangs the garments “G” and/or other hunting accessories upon a drying rack or from a clothesline, which enables such garments or accessories to air-dry. As desired, the user re-submerges or retreats garments “G” and/or other hunting accessory in an odor absorbing agent 12 and water solution, until the desired effect is achieved.

Referring now to FIGS. 7 and 9, as desired, a user uses odor absorbing substance delivery device 10F to apply odor absorbing agent 12 to e.g. garment “G” and/or other hunting accessories. As illustrated in FIG. 9, a user suspends, for example, garment “G” from clothesline “CL” by way of clothespins “CP.” Then, the user loads a desired volume of odor absorbing agent 12 into jar 82, attaches jar 82 to jar-cover 86, and connects hose “H” to coupling mechanism 92. Then, the user activates the device, whereby water from the pressurized water source travels through hose “H,” into and through jar 82 where it mixes with odor absorbing agent 12, and exits through nozzle 88 while carrying odor absorbing agent 12 therewith and/or therein. In other words, the user sprays the garment “G” with water and odor absorbing agent 12. The garment “G” is permitted to air dry, thence, as desired, a user can re-spray the garment “G” and/or other hunting accessory, until the desired effect is achieved.

Referring now to FIGS. 10 and 11, as desired, a user uses odor absorbing substance delivery device 10G to apply odor absorbing agent 12 to e.g. garment “G” and/or other hunting accessories. As illustrated in FIG. 11, similar to that of FIG. 9, a user suspends, for example, garment “G” from clothesline “CL” by way of clothespins “CP.”

Then, the user orients aerosol can 100, namely the exit orifice of nozzle 105, facing garment “G”. Then, the user depresses nozzle 105, whereby the pressurized propellant 120 drives odor absorbing agent 12 of aerosol can 100, onto garment “G”. The user continues to spray garment “G”, or repeatedly sprays garment “G”, until the desired effect is achieved.

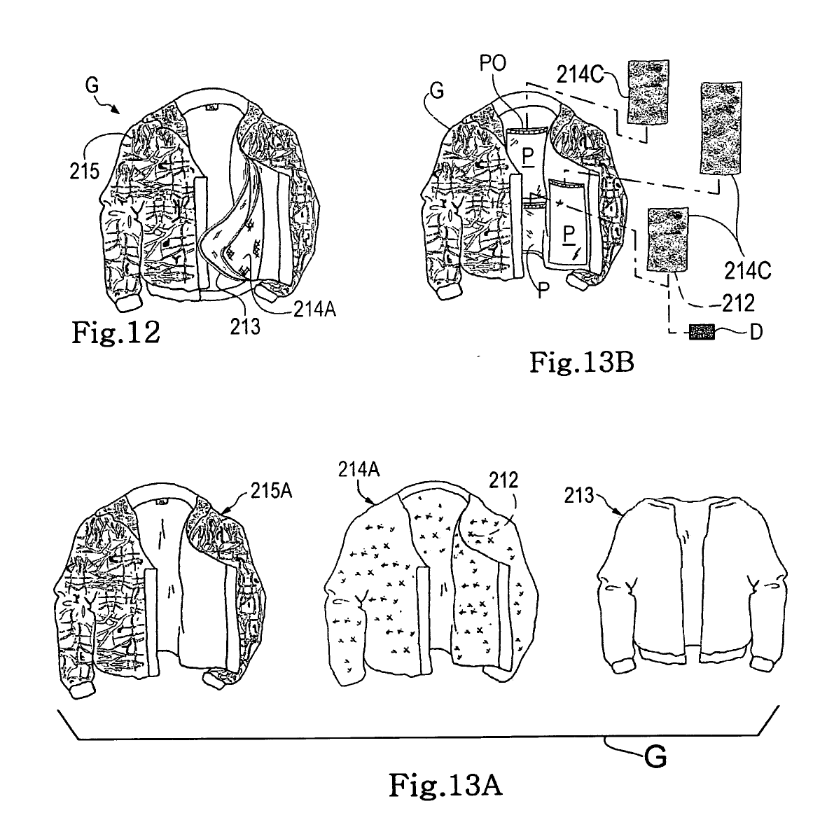

As illustrated in FIGS. 12, 13A, and 13B, in some embodiments, garment “G” includes at least one removable portion thereof. Such removable portion is adapted and configured to accept odor absorbing agent 12, thereupon or therein. The removable portion of garment “G” enables a user to, as desired, reactivate, recharge, or otherwise restore the odor absorbing and/or masking qualities and characteristics of garment “G” by applying or reapplying, the desired odor absorbing substances, deodorizers or other odor masking agents, collectively referred to as odor absorbing agents.

As described in greater detail elsewhere herein, in some embodiments, the removable portion of garment “G” enjoys generally the same temporal use life as compared to the remainder of the garment. In some embodiments, the removable portion of garment “G” enjoys a generally shorter temporal use life as compared to the remainder of garment “G”, whereby the removable portion of garment “G” is, for example, adapted and configured for a single use, optionally relatively few reactivation cycles such as less than 20 reactivation cycles, optionally relatively many reactivation cycles such as greater than 20 reactivation cycles, and/or others as desired.

In other words, garment “G” includes inner layer 213, odor absorbing article 214A as an intermediate layer, and outer layer 215A. Referring to FIGS. 12 and 13A, inner layer 213 is, for example, a coat liner i.e. a coat inner liner. Layer 213, in the complete assemblage of garment “G” is adjacent the body of the user, when the user wears garment “G.” Layer 213 provides, as desired, insulating functionality to the user, and/or a physical barrier between the user and odor absorbing article 214A.

Odor absorbing article 214A is adapted and configured to hold a sufficient amount of odor absorbing agent 12 thereupon and/or therein to provide the desired level of odor absorbing functionality. Thus, odor absorbing article 214A is made from materials which are capable of holding odor absorbing agent 12 thereupon or therein. This is optionally done with materials which have a particularly suitable affinity for the odor absorbing agent 12 and/or possess physical characteristics which enable the odor absorbing agent 12 to rest upon, in, or otherwise become lodged upon, or in, or otherwise held by, the material of article 214A.

As one example, odor absorbing article 214A is made of one or more materials which are sufficiently porous, fibrous, permeable, stringy, matted, woven, or otherwise suitably capable of trapping or holding various particles of the odor absorbing agent 12 in e.g. interstices thereof.

Odor absorbing article 214A can be made from material(s) which, themselves, have at least some odor absorbing and other desirable qualities. Such materials include various ones which include, as a component thereof, e.g. silicon-based nanophase substances such as an aerogel or an aerogel containing material. One suitable aerogel containing material is sold under the trademark Pristina™ which is available from the TAASI Corporation of Delaware, Ohio.

As desired, and as described in greater detail elsewhere herein, a user can reapply odor absorbing agent 12 to odor absorbing article 214A using any of a variety of suitable methods, techniques, and devices.

As shown in FIG. 13A, in some embodiments, odor absorbing article 214A is generally similar in size and configuration to the size and configuration of inner layer 213. However, odor absorbing article 214A is preferably at least nominally larger than inner layer 213, whereby inner layer 213 can be suitably housed within odor absorbing article 214A. Thus, in the complete assemblage of garment “G,” the outer surface of inner layer 213 interfaces with the inner surface of odor absorbing article 214A. The outer surface of odor absorbing article 214A interfaces with the inner surface of outer layer 215.

Outer layer 215 is, for example, an outer jacket or coat layer or shell. Accordingly outer layer 215 is typically the visible portion of garment “G.” Thus, the outer surface of outer layer 215, as desired, includes an e.g. camouflage pattern or other desired indicia.

The various layers of garment “G,” namely inner layer 213, odor absorbing article 214A, outer layer 215, and/or other layers with can be incorporated into garment “G,” are retained in an acceptable positional relationship with respect to each other in any of a variety of suitable e.g. connecting methods and devices. As one example, ones of inner layer 213, odor absorbing article 214A, outer layer 215, and/or other layers which can be incorporated into garment “G,” are held to respective other ones of the layers by way of zippers which, for example, zip the outer edges of the various layers to each other adjacent the main closure zipper, or elsewhere e.g. adjacent the sleeve ends, neck opening, bottom edge, and/or elsewhere as desired.

As another example, ones of inner layer 213, odor absorbing article 214A, outer layer 215, and/or other layers which can be incorporated into garment “G,” are held to respective other ones of the layers by way of various hook and loop fasteners, such as those available under the trademark VELCRO®, available from Velcro Industries B.V., which, for example, fasten corresponding portions of the layers to each other. Exemplary and non-limiting suitable locations of layer attachment are e.g. at or adjacent the various outer edges of the various layers such as adjacent the main closure zipper, or elsewhere e.g. adjacent the sleeve ends, neck opening, bottom edge, and/or elsewhere as desired.

There can also be mentioned other suitable ways of attaching various ones of the garment layers to respective other ones of each other, which include, but are not limited to, buttons, frictional engagement, and others.

It should be noted that the modularity of garment “G” (of FIGS. 12 and 13A) provides the user with several garment use options. As one example, a user can substitute an alternative outer layer 215 for that which is currently used. In other words, if a user is utilizing a first outer layer 215 having a first camouflage pattern, the user can remove such outer layer and replace it for a second outer layer 215 having a second, different, camouflage layer.

Accordingly, the user can acquire a plurality of outer layers 15, such as a green/brushy-appearance camouflage outer layer, a white/snowy-appearance camouflage outer layer, a brown/sandy-appearance camouflage outer layer, a blaze-orange or other conspicuously colored outer layer, and/or others, in lieu of acquiring a plurality of individual coats, jackets, or other garments, for the aforementioned patterns.

Thus, the modularity of the other layers of garment “G” further add to the variety of suitable uses for garment “G.” As another example, there can be provided different inner layers 13 having different weights, thicknesses, and insulating characteristics, whereby the user selects and uses the appropriate inner layer 213 which corresponds to the intended use environment.

Garment “G” enables a user to make use of the odor absorbing qualities of odor absorbing layer 214A while mitigating direct user-to-odor absorbing agent 12 contact and thus remaining relatively clean. Garment “G” also enables a user to make use of the odor absorbing qualities of odor absorbing layer 214A without compromising the visual integrity of the camouflage or other indicia on the outer surface of outer layer 215.

In other words, during use of garment “G”, any airborne or other particles of the odor absorbing agent 12 are generally retained within the interior structure of the garment. Namely, any airborne or other particles of odor absorbing agent 12 are generally physically restrained by, for example, the outer surface of inner layer 213 and the inner surface of outer layer 215, whereby the odor absorbing agent 12 is generally not in direct contact with the user or the outermost visible surfaces of garment “G.”

Thus, generally the only time that the user is directly exposed to the odor absorbing agent 12 is when the user chooses to remove odor absorbing layer 214A from garment “G” and reapply odor absorbing agent 12 thereto, which is explained in greater detail elsewhere herein.

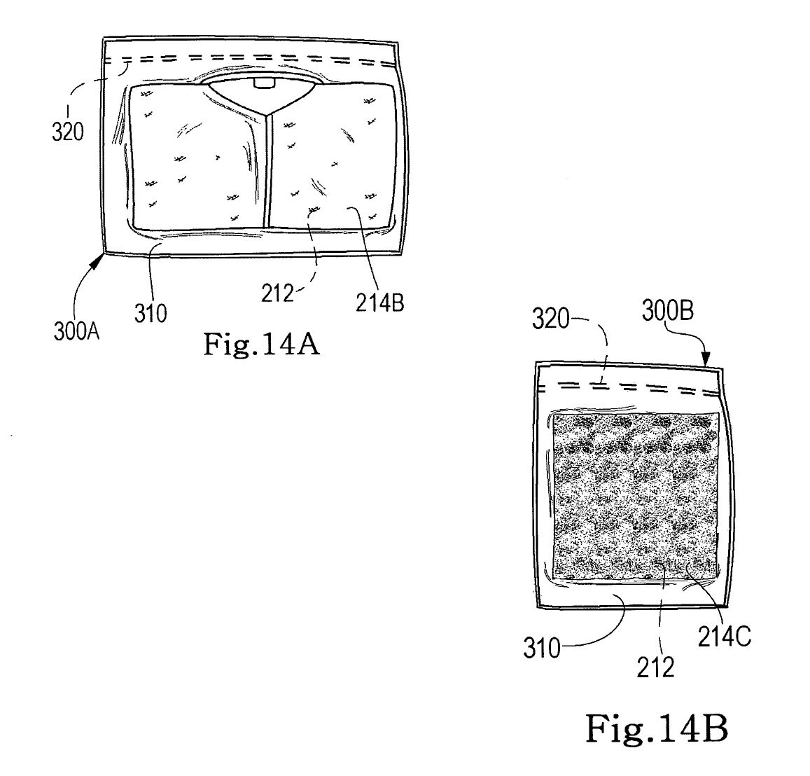

Referring now to FIGS. 13B, 14A, and 14B, in some embodiments, the odor absorbing substrate or article is adapted and configured for a generally defined, limited, use-life. In other words, in some embodiments, the odor absorbing substrate or article is disposable and relatively less suitable for repeated recharging with the odor absorbing agent or having particles of the odor absorbing agent 12 reapplied thereto.

In such embodiments, such as disposable layer 214B, disposable panels 214C, and others, the odor absorbing agent 12 is pre-applied to the substrate or article, whereby the user merely inserts the substrate or article into, or otherwise assembles the substrate or article to, the garment or other article of clothing, or other game hunting or game watching accessory.

Referring to FIGS. 12, 13A, and 13B, as desired, disposable layer 214B is used in lieu of or in addition to odor absorbing layer 214A. Disposable layer 214B is also an odor absorbing article and is sized and configured similar to e.g. odor absorbing layer 214A. In other words, disposable layer 214B looks like an upper torso garment such as a shirt, coat, jacket, or coat or jacket liner. However, whereas odor absorbing layer 214A is made from typical textile materials, disposable layer 214B is, optionally, made from relatively less of those materials, e.g. disposable layer 214B is a relatively thinner layer as compared to odor absorbing layer 214A.

In the alternative, disposable layer 214B is made from other materials which are less frequently used as clothing structures, yet which are which are relatively more economically suitable for a relatively short use life, which ultimately concludes with disposal of the layer 214B. Exemplary of such materials are those with e.g. paper based layers or components, such as various tissue based materials and various non-woven layers made of natural or synthetic fibers, or mixtures of natural and synthetic fibers. Any tissue based materials, or any other materials which might prove relatively noisy during flexing, bending, or other manipulations, are not overly noisy so as to e.g. alert game during hunting or observation activities. In other words, although tissue based and other materials might be relatively more noisy during user movement as compared to more typical textile materials, such tissue based or other materials are preferable still suitably quiet for the intended use environment.

Various odor absorbing agents 12, such as various non-carbon based and carbon based odor absorbing agents, perform more efficiently in relatively low moisture environments. Accordingly, when utilizing various e.g. carbon based products as odor absorbing agents 12, the user, as desired, utilizes a removable layer which includes an e.g. gas permeable and liquid impermeable membrane or layer, communicating therewith. As one example, in some embodiments, the tissue or synthetic non-woven or mixture based removable layer includes various ones of configurations such as (i) poly tissue construction having a polymeric film or fabric layer and a tissue layer, (ii) tissue poly tissue construction having first and second tissue layers on opposing sides of a polymeric film or fabric layer, and/or others.

The polymeric film or fabric layer is adapted and configured to provide, for example, a moisture barrier between the user and the odor absorbing article, whereby the amount of e.g. user perspiration and/or other moisture from the user, which ultimately interacts with the odor absorbing agent 12 is mitigated, while still permitting user odors to pass therethrough and thus interact with odor absorbing agent 12. Accordingly, as desired, the odor absorbing agent 12 is maintained in a suitably dry use environment to preserve the integrity of its odor absorbing qualities, characteristics, and functionality.

Optionally, various other components of garment “G” include a polymeric film or fabric component or layer, such as inner layer 213, and adjacent layer, and/or others, to effectuate a user moisture transfer mitigating environment which keeps odor absorbing agent 12 relatively dry from user based moisture, when using odor absorbing agents 12 which are detrimentally effected by relatively high-moisture use environments. Accordingly, as desired, a polymeric layer, poly tissue, or tissue poly tissue layer, is placed outside of the odor absorbing layer (not illustrated), whereby the polymeric film or fabric mitigates the amount of ambient moisture which interacts with the odor absorbing agent 12. Suitable locations for such polymeric layer include, for example, on or adjacent outer layer 215, or elsewhere between the odor absorbing article and the ambient air.

Referring to FIG. 13B, in some embodiments, garment “G” utilizes one or more odor absorbing articles, optionally disposable odor absorbing articles, which are removably placed into various compartments within the garment. Namely, garment “G” includes one or more pockets “P”, each of which is a compartment adapted and configured to removably house an odor absorbing article.

As one example, each pocket “P” is for example a cloth panel which is attached, at its perimeter, to portions of garment “G”, such as the inner surface, the outer surface, a surface of a removable coat liner or layer, or other suitable pocket-type mounting surfaces. Each pocket “P” includes a pocket opening “PO” which enables a user to insert an odor absorbing article into and remove an odor absorbing article from such pocket, as desired. Pocket opening “PO” is illustrated as an opening at the uppermost edge of pocket “P” although it is appreciated and fully realized that such pocket opening “PO” can be anywhere upon pocket “P” which enables insertion and withdrawal of an odor absorbing article, i.e. at or adjacent ones of the lateral edges, or elsewhere.

Referring now to FIGS. 13A and 14B, exemplary of such odor absorbing article for use with pocket “P” is disposable panel 214C. Disposable panel 214C is an odor absorbing article, which utilizes an odor absorbing agent 12 applied thereto. Namely, disposable panel 214C is a generally flexible, planar, sheet-like structure which is impregnated with odor absorbing agent 12 and/or otherwise has odor absorbing agent 12 applied thereupon or therein.

In some embodiments, like disposable layer 214B (FIG. 14A), disposable panel 214C is adapted and configured for a generally defined, limited, use-life. In other words, disposable panel 214C does not require repeated recharging with odor absorbing agent 12, i.e. does not require reapplication of odor absorbing agent 12 thereto; rather, at the end of the use life of disposable panel 214C, the user discards disposable panel 214C. Optionally, disposable panel 214C (or any other disposable portions of garment “G” is adapted and configured for a use life with is relatively shorter than the use life of the remainder of garment “G”, whereby disposable panel 214C is adapted and configured to survive less than twenty recharge cycles, more than 20 recharge cycles, or others.

Disposable panels 214C can be made from any of a variety of materials capable of suitably holding a sufficient amount of odor absorbing agent 12 thereupon and/or therein. In other words, disposable panel 214C is made from e.g. materials which are capable of holding particles of the odor absorbing agent 12 thereupon or therein.

This is done by utilizing, for example, materials which have a particularly suitable affinity for the odor absorbing agent 12 and/or possess physical characteristics which enable the odor absorbing agent 12 to rest upon, in, or otherwise become lodged upon, in, or otherwise held by the material of disposable panel 214C. As one example, disposable panel 214C is made of one or more materials which are sufficiently porous, fibrous, permeable, stringy, matted, woven, or otherwise suitably capable of trapping or holding particles of the odor absorbing agent 12 in e.g. interstices thereof.

In addition to removably holding ones of disposable panels 214C, in some embodiments, each pocket “P” is adapted and configured to hold, for example, desiccant or a package of desiccant therein. Accordingly, a respective pocket “P” can hold one or more disposable panels 214C and desiccant “D” therein. In some embodiments, desiccant “D” is retained in pockets which are separated from, yet preferably adjacent, the pockets “P” which retain the disposable panels 214C.

Desiccant “D” includes any of a variety of suitable desiccant substances, materials, and/or products which relatively reduce the amount of airborne moisture interfacing with odor absorbing agent 12 by e.g. drawing such moisture thereto. Exemplary of such desiccant substances are any of a variety of substances which are suitably hygroscopic to produce the desired results, which include, but are not limited to, non-indicating silica gel, indicating silica gel, and/or others.

In yet other embodiments, desiccant “D” is a silicon-based nanophase material such as an aerogel or an aerogel containing material, which can also be used as the odor absorbing agent 12 itself.

When using embodiments such as those of FIGS. 13B, 14A, and 14B, the user uses garment “G” until such time as ones of disposable layer 214B and disposable panel 214C, collectively referred to as disposable odor absorbing article 214B, 214C, sufficiently lose their odor absorbing effectiveness.

At that time, when the disposable odor absorbing article 214B, 214C is no longer sufficiently effective, the user removes such disposable odor absorbing article 214B, 214C, discards or disposes of it. Then, the user installs a new disposable odor absorbing article 214B, 214C into garment “G” to restore the garments' odor absorbing qualities, characteristics, and functionality.

Since the odor absorbing agent 12 is pre-applied, before reaching the end-user, odor absorbing articles 214B and 214C are preferably packaged, housed, stored, and/or otherwise enclosed in a manner which suitably preserves the integrity of the odor absorbing agents' odor absorbing qualities, characteristics, and functionality, prior to use.

Referring now to FIGS. 14A and 14B, packaged odor absorbing article 100A and packaged odor absorbing article 100B illustrate exemplary suitable methods of storing odor absorbing articles 214B, 214C, respectively.

Each of packaged odor absorbing articles 300A, 300B includes the respective odor absorbing article 214B, 214C, and sealed enclosure 310. In some embodiments, each sealed enclosure 310 includes a single odor absorbing article 214B, 214C, optionally, a plurality of odor absorbing articles 214B, 214C.

Sealed enclosure 310 is preferably a polymeric film or other polymeric enclosure with sufficiently low gas transmissibility characteristics to mitigate the amount of air, ambient or otherwise, and corresponding odors associated with such air, exposed to the odor absorbing agent 12 of odor absorbing articles 214B, 214C. In some embodiments, sealed enclosure 310 is vacuum sealed which further mitigates the amount of odors which the odor absorbing agent 12 is exposed to prior to the intended utilization of the odor absorbing articles 214B, 214C.

In some embodiments, sealed enclosure 310 includes an easy-opening mechanism such as line of weakness 320. Line of weakness 320 enables a user to tear open sealed enclosure 310 along such line of weakness, and thus relatively simplifies the process and procedure for removing the odor absorbing article 214B, 214C from the respective sealed enclosure 310.

Preferably, components of odor absorbing substance delivery devices 10A, 10B, 10C, 10D, 10E, 10F and 10G are made of materials which resist corrosion, and are suitably strong and durable for normal extended use. Those skilled in e.g. the packaging art are well aware of certain, for example, polymeric materials which possess such desirable qualities, and appropriate methods of forming such materials.

Polymeric materials suitable for components of odor absorbing substance delivery devices 10A, 10B, 10C, 10D, 10E, 10F and 10G, such as various ones of bottle 20, box-container 30, bag 50, container 60, hose-end sprayer 60, and/or others, are various polymeric compounds, such as for example and without limitation, various of the polyolefins, such as a variety of the polyethylenes, e.g. high density polyethylene, or polypropylenes. There can also be mentioned as examples such polymers as polyvinyl chloride and chlorinated polyvinyl chloride copolymers, various of the polyamides, polycarbonates, and others.

For any polymeric material employed in structures of the invention, any conventional additive package can be included such as, for example and without limitation, slip agents, anti-block agents, release agents, anti-oxidants, fillers, and plasticizers, to control e.g. processing of the polymeric material as well as to stabilize and/or otherwise control the properties of the finished processed product, also to control hardness, bending resistance, and the like.

Common industry methods of forming such polymeric compounds will suffice to form non-metallic components of odor absorbing substance delivery devices 10A, 10B, 10C, 10D, 10E, 10F and 10G. Exemplary, but not limiting, of such processes are the various commonly-known plastics converting processes.

Those skilled in the art will now see that certain modifications can be made to the apparatus and methods herein disclosed with respect to the illustrated embodiments, without departing from the spirit of the instant invention. And while the invention has been described above with respect to the preferred embodiments, it will be understood that the invention is adapted to numerous rearrangements, modifications, and alterations, and all such arrangements, modifications, and alterations are intended to be within the scope of the appended claims.

To the extent the following claims use means plus function language, it is not meant to include there, or in the instant specification, anything not structurally equivalent to what is shown in the embodiments disclosed in the specification.

Having thus described the invention, what is claimed is:

1. A method of suppressing odor and visually camouflaging, the method comprising:
(a) applying a dry powder form of an odor suppressing agent to hunting garments; and
(b) applying a dry powder form of an odor suppressing agent to exposed skin surfaces of a user.

2. A method as in claim 1 wherein the odor suppressing agent is applied by dispensing from a bottle.

3. A method as in claim 1 wherein such dry powder form of the odor suppressing agent is applied to such hunting garments by sealing the garments in a bag containing the agent and agitating the contents of the bag.

4. A method as in claim 1 further including applying a plurality of relatively differently colored odor suppressing agents to such hunting garments.

5. A method as in claim 4 wherein such hunting garments define a camouflage pattern.

6. A method as in claim 4 wherein such hunting garments define a camouflage pattern and such differently colored odor suppressing agents are applied to the garments in a manner that visually corresponds to the camouflage pattern.

7. A method of generally simultaneously camouflaging and suppressing odor, the method comprising:
(a) applying an odor suppressing agent, carried in a liquid carrier, to hunting garments; and
(b) applying a dry powder form of an odor suppressing agent to exposed skin surfaces of a user.

8. A method as in claim 7 wherein the liquid carrier is an aqueous solution.

9. A method as in claim 7 wherein the method further includes forming a solution within a container, the solution including the odor suppressing agent, thence placing the hunting garments into the solution and agitating the garments therein.

10. A method as in claim 7 wherein the method further includes forming a solution within a container, by admixing an odor suppressing tablet and a volume of liquid together, thence placing the hunting garments into the solution and agitating the garments therein.

11. A method of generally simultaneously suppressing odor and visually camouflaging, the method comprising:
(a) applying a first odor absorbing agent to an exposed skin surface of a user; and
(b) applying a second odor absorbing agent to an exposed skin surface of a user,
wherein said first and second odor absorbing agents define first and second colors which differ from each other.

12. A method as in claim 11 wherein said first color defines a green color and said second color defines a brown color.

13. A method as in claim 11 wherein said first color defines a green color and said second color defines a black color.

14. A method as in claim 11 wherein said first color defines a relatively light color and said second color defines a relatively dark color.

15. A method as in claim 11 wherein ones of said first and second odor absorbing agents are applied with a brush.

16. A kit for generally simultaneously camouflaging and suppressing odor, the kit comprising:
(a) a first odor absorbing agent which defines a first color;
(b) a second odor absorbing agent which defines a second, different, color; and
(c) at least one delivery device adapted and configured to deliver ones of said first and second odor absorbing agents from such device to a user.

17. A kit as in claim 16, wherein said delivery device comprises at least one bottle.

18. A kit as in claim 16, wherein said delivery device comprises a compressed form of at least one of said first and second odor absorbing agents.

19. A kit as in claim 18, wherein said compressed form of at said least one of said first and second odor absorbing agents generally defines a tablet configuration.

20. A kit as in claim 16, wherein at least one of said first and second odor absorbing agents is housed in a container and defines a predetermined volume of the agent.
